(19) United States
(12) Patent Application Publication
(10) Pub. No.: US 2026/0262980 A1
(43) Pub. Date: Sep. 10, 2026

(54) PRIVACY-PRESERVING ARTIFICIAL INTELLIGENCE FOR EMPLOYEE DEPRESSION DETECTION

(71) Applicant: Dell Products L.P., Round Rock, TX (US)

(72) Inventors: Zijia Wang, London (GB); Mustafa Al-Bado, Cork (IE); Min Gong, Shanghai (CN)

(21) Appl. No.: 19/074,856

(22) Filed: Mar. 10, 2025

(51) Int. Cl. A61B 5/16; G06F 21/62; G06V 10/774; G06V 40/16

(52) U.S. Cl. CPC A61B 5/165; G06F 21/6245; G06V 10/774; G06V 40/172

(57) ABSTRACT

The disclosure introduces a system that integrates a learnable optical model with a multi-task deep network to detect depression while preserving user privacy. The system utilizes an optimized lens that captures images obscuring identity information while retaining features for depression detection. A feature focus network is configured to extract and analyze facial cues indicative of depression through a progressive learning strategy. This strategy incrementally trains the deep network in stages, focusing first on identity obfuscation, followed by emotional feature extraction, and depression-specific feature extraction. A multimodal transformer fuses emotional and depression features to produce an accurate depression score. The system is co-optimized through an end-to-end joint optimization process, ensuring that captured images are privacy preserving images that are effective for depression detection.

### TECHNOLOGICAL FIELD OF THE DISCLOSURE

Embodiments disclosed herein generally relate to user privacy and user health. More particularly, at least some embodiments relate to systems, hardware, software, computer-readable media, and methods for protecting user privacy and user health and more particularly to monitoring and/or monitoring user health while protecting or preserving user privacy.

### BACKGROUND

Entities have a significant interest in the health of their employees at least because unhealthy employees are less productive. To improve employee health, some entities attempt to monitor their employees for various health issues including mental health issues. An employee experiencing depression, if unknown, may not receive the support they need, be less productive, and adversely impact the work environment. The goal of helping employees be healthy and productive, however, may be impacted by a competing need to preserve employee privacy. Traditional methods for monitoring employee health often compromise their privacy and may lead to breaches of confidentiality and the misuse of sensitive personal information.

### BRIEF DESCRIPTION OF THE DRAWINGS

In order to describe the manner in which at least some of the advantages and features of one or more embodiments may be obtained, a more particular description of embodiments will be rendered by reference to specific embodiments thereof which are illustrated in the appended drawings. Understanding that these drawings depict only typical embodiments and are not therefore to be considered to be limiting of the scope of this disclosure, embodiments will be described and explained with additional specificity and detail through the use of the accompanying drawings, in which:

FIG. 1 discloses aspects of a privacy lens system configured to generate privacy preserving images;

FIG. 2 discloses aspects of a deep network configured to monitor user health;

FIG. 3 discloses aspects of training a deep network for monitoring user health;

FIG. 4 discloses aspects of monitoring user health; and

FIG. 5 discloses aspects of a computing device, a computing system, or a computing entity.

### DETAILED DESCRIPTION OF SOME EXAMPLE EMBODIMENTS

Embodiments disclosed herein generally relate to artificial intelligence based monitoring. More particularly, at least some embodiments relate to systems, hardware, software, computer-readable media, and methods for monitoring user health while preserving user privacy.

Embodiments of the invention are discussed in the context of monitoring users to detect health concerns and issues including depression. Aspects of monitoring their health may include taking remedial or proactive actions to address the issue or underlying causes, assisting users in seeking treatment, or the like. More specifically, embodiments of the invention relate to monitoring and/or detecting health issues, such as depression, while preserving user privacy. However, embodiments of the invention may be implemented in the context of other health concerns or other issues. For example, embodiments of the invention may be used to detect characteristics from scenes from which images are taken, whether the scenes include users or not, while preserving privacy related to the scene.

The detection of depression using machine learning and deep learning techniques often rely on analyzing textual data from social media or clinical interviews. These approaches, even if effective, raise privacy concerns because they involve the collection and analysis of personal data.

In some instances, facial expressions and physiological signals may be used to detect depression. For example, coding systems may be used to identify depression-related facial movements. An approach combining audio, visual, and physiological data may improve the accuracy of depression detection. However, these methods face challenges in protecting user privacy at least because they require identifiable facial images and other personal data.

To protect privacy in the context of machine learning, techniques such as differential privacy and federated learning may be employed. Differential privacy adds noise to the collected data to prevent the identification of individuals, while federated learning allows models to be trained on decentralized data without transferring local data to a central server. Despite these advancements, applying these techniques to depression detection remains challenging due to the need for high accuracy and the complexities associated with the data and with detecting health issues such as depression.

Embodiments of the invention address at least these problems and may include a system with a learnable optical model (e.g., a learnable lens) with a deep network to detect depression while preserving user privacy. The system utilizes a lens that captures images in a manner that obscures identity while retaining relevant features for depression detection. In one example, a feature focus network (FFNet) is configured to extract and analyze facial cues (features) indicative of depression through a progressive learning strategy. Embodiments of the invention incrementally train the feature focus network in stages while optimizing the learnable lens at the same time. Embodiments of the invention focus first on identity obfuscation, which is followed by emotional feature extraction, and finally, depression-specific feature extraction. Embodiments of the invention further include a multimodal transformer that fuses the extracted emotional and depression features, to produce a depression score. The system is co-optimized through an end-to-end joint optimization process, thereby ensuring that images remain privacy-protective while being effective for depression detection.

Embodiments of the invention allow an entity to proactively support employee (user) mental health in a confidential manner. This advantageously enhances employee wellbeing, productivity, and overall job satisfaction. Furthermore, the adaptability of privacy-preserving health monitoring enables scalability and privacy-focused solutions for various purposes across various sectors.

In one example, a deep network architecture is configured to extract features for depression detection from privacy preserving images. A multimodal transformer is further configured to fuse emotional and depression features, leading to a more accurate final depression score.

Embodiments of the invention generally relate to a learnable privacy-preserving lens that obfuscates identity features while preserving emotion and/or depression-related features.

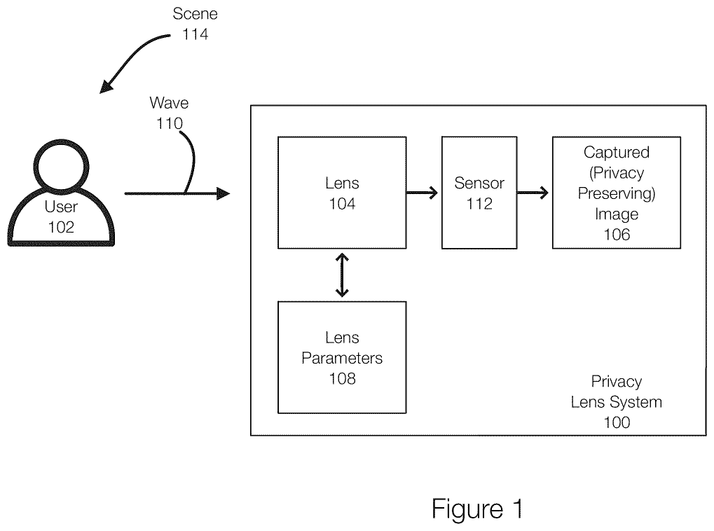

FIG. 1 discloses aspects of a learnable privacy-preserving lens includes in a lens system. FIG. 1 illustrates a privacy lens system 100 (system 100) that is configured to generate or a captured or privacy preserving image 106 (image 106). More specifically, the system 100 is configured to capture images, represented by the image 106, that obscure identity-related features of a user 102 while preserving structural details or features used to detect depression or other characteristic or issue.

In this example, the system 100 is discussed in the context of a wave-based image formation process. Thus, a wave 110 of a scene, such as the user 102, is received by the system 100. The system 100 may include a lens 104, which is representative of a physical lens having a certain surface of a point spread function (PSF) in one example. In other words, the lens 104 may by a physical learnable lens or a model. In one example, the PSF is determined by a surface profile of the lens 104, which may be modeled using Zernike polynomials in one example.

The system 100 or more specifically the lens 104 is trained or configured to obscure identity-features while ensuring that other features relevant for emotional or depression detection, are not obscured or removed. The lens 104 may be configured or trained, in one example, by optimizing the lens parameters 108.

In this example, a wave 110 of a scene 114 that includes a user 102 is received by the lens 104. An output of the lens 104 is determined by the parameters 108 and is detected by the sensor 112. The image 106 is thus obtained from the sensor 112. The lens parameters 108 can be learned or trained such that identity features of the user 102 are obscured while features for detecting depression are not obscured or are obscured to a lesser degree.

The captured image I_{c}(x, y) is computed as Z or:

$${I}_{c} ( x , y ) = ( {I}_{λ} * {P}_{λ} ) ( x , y ) {κ}_{C} ( λ ) d λ + η .$$

In this example, I_{λ} represents the original scene, P_{λ} represents the PSF, κ_{c}(λ) represents the sensitivity of the sensor 112 to wavelength λ, and η represents Gaussian noise. The phase change induced by the lens is given by:

$$Φ ( {x}^{′} , {y}^{′} ) = \frac{2 π Δ n}{λ} H ( {x}^{′} , {y}^{′} ) .$$

In this example, Δn is a refractive index difference, λ is the wavelength, and H(x′, y′) is the surface profile of the lens 104 expressed as a sum of Zernike polynomials as follows:

$$H ( {x}^{′} , {y}^{′} ) = {\sum }_{j = 1}^{q} {α}_{j} {Z}_{j} ( {x}^{′} , {y}^{′} ) .$$

In this example, αⱼ represents the coefficients of the Zernike polynomials Zⱼ.

The learnable lens 104 may be optimized (or co-optimized) alongside a deep network to ensure that the image 106 obscures identity while retaining key features necessary for accurate depression detection. The loss function for this optimization includes a combination of Mean Squared Error (MSE) loss, identity obfuscation loss, and landmark preservation loss as follows in one example:

$${L}_{i} = α {L}_{v} + β {L}_{i d} + γ {L}_{l m} .$$

In this example, Lᵥ is a visual degradation loss, L_{id} is an identity obfuscation loss, and Lₗₘ is a landmark preservation loss. In one example, the visual degradation loss may be used to ensure that the image remains coherent while undergoing a transformation. The identity obfuscation loss helps ensure that features used for identity are removed during the transformation. The landmark preservation loss helps ensure that certain features remain in the correct positions after transformations. Minimizing these losses is an example of ensuring that the system generates a captured image 106 of the user 102 that obscures identity while retaining features that may be used for other purposes such as emotional detection, depression detection, or the like.

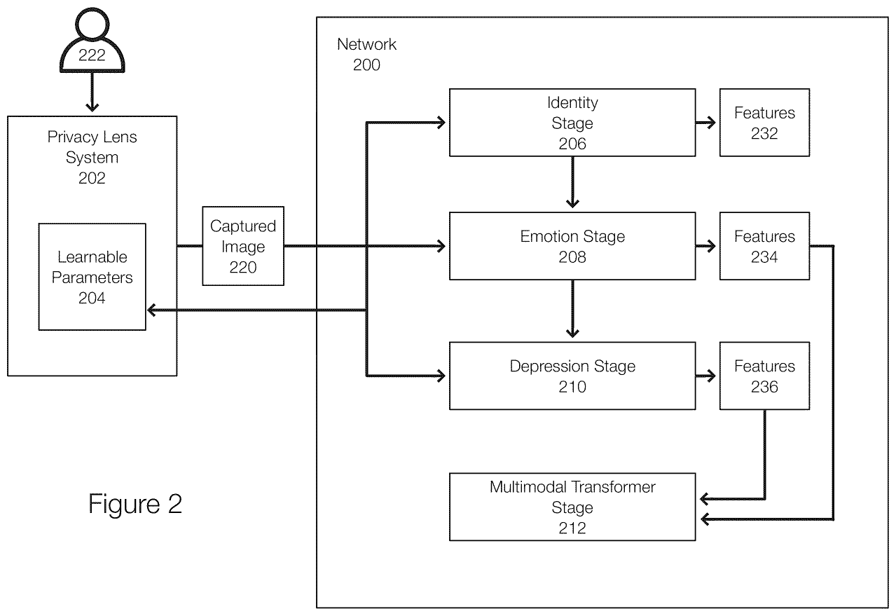

FIG. 2 discloses aspects of a feature focus network configured to detect a health issue or other characteristic. FIG. 2 discloses aspects of training or optimizing a network 200 (e.g., an FFNet) while also optimizing the privacy lens system 202 (or the lens or lens parameters), which is an example of the system 100.

The network 200 may be trained in stages and the lens system 202 (or the lens parameters) may be optimized at the same time along with the stages. For example, an image 220, which may be a blurred or privacy preserving image generated by a lens representing a PSF function having learnable parameters 204, may be generated by the privacy lens system 202 and input to the network 200.

More specifically, the captured image 220 may be input to the identity stage 206 initially. The identity stage 206 may include an identity model that is pretrained to recognize an identity of a user 222 from the captured image 220. The captured image 220 may be use to further train the identity model. In one example, the features 232 of the captured image 220 required to determine the identity of the user 222 can be identified using a loss function and/or backpropagation. The features 232 may be used to adjust the learnable parameters 204. In other words, the identity stage 206 allows the learnable parameters 204 to be learned or adapted such that identity of the user 222 is obscured in the captured image 220, while still allowing the identity stage 206 to determine an identity of the user 222. This protects privacy of the user 222 (e.g., the image is not transmitted in the clear) while allowing the health status of the user 222 to be determined in a confidential and privacy protecting manner.

Once the parameters 204 are updated by the identity stage, the captured image 220 may (or may not) be updated based on the updated parameters 204 and input to an emotion stage 208. The emotion stage 208 may include an emotion model configured to extract emotional features 234 used for or relevant for detecting emotion. In one example, a cross-entropy loss function may be employed in the context of emotion classification. Stated differently, the emotion model may be configured to classify the capture image 220 with respect to emotion classes. The emotion determined by the emotion stage 208 may be the class with a highest probability. The features 234 can be correlated with the determined emotion class.

The cross-entropy loss may be represented as:

$${L}_{e} = - {\sum }_{c} \text{?} log ( {p}_{c} ) .$$

In this example, [symbol] is a true label and pc is a predicted probability for emotion class c.

The emotion stage 208 may be configured to extract emotional features 234 or, more specifically, features 234 of the captured image 220 that are related to detecting emotion or depression.

This allows the learnable parameters 204 to be updated based on a loss or features 234 extracted by the emotion stage 208.

In one example, the capture image 220 (or more specifically the learnable parameters 204) is updated based on the emotion stage 208 and the features 234 and the updated captured image 220 provided to the depression stage 210. The depression stage 210 may include a depression model configured to identify features 236. More specifically, the features 236 may include depression-specific features that are identified by the depression stage 210 using the previously learned emotional features 234 as priors. Thus, the features 234 are priors and may provide context or constraints to the depression stage 210 such that the features 236 can be extracted from the captured image 220. The features 236 may also be used to update the learnable parameters 204.

The network 200 may include a multimodal transformer stage 212. The stage 212 may include a multimodal transformer that employs cross-modality attention to merge the emotional features 234 and the depression features 236. A multimodal transformer may be configured to identify relationship[s between different data (e.g., the features 234 and the features 236) to better understand the relationships that may be present between these different data. Fusing the features 236 and 236 allows a multimodal transformer to generate a depression score that is improved compared, in one example, to a depression score based on the features 236 alone.

The stage 212, more specifically, merges the emotion features 234 (ZE) and the depression features 236 (Z_{D}) as:

$${Z}_{E \to D} = Attention ( {Q}_{E} , {K}_{D} , {V}_{D} ) and$$

$${Z}_{D \to E} = Attention ( {Q}_{D} , {K}_{E} , {V}_{E} ) and$$

In this example, Q, K, and V represent, respectively, the query, key, and value matrices of the multimodal transformer. The final fused features are obtained by concatenating the outputs from both directions as follows:

$${Z}_{fusion} = [ {Z}_{E \to D} ; {Z}_{D \to E} ] .$$

In one example, the fused features are passed through a final dense layer to predict a depression score. The loss function used in the stage 212 is an MSE between the predicted and true depression scores as follows:

$${L}_{S} = \frac{1}{n} {\sum }_{i = 1}^{n} {( {S}_{i} - \text{?} )}^{2} .$$

In this example, Sᵢ is a true score and [symbol] is a predicted score.

As illustrated in FIG. 2, training the network 200 and the privacy lens system 202 is performed in an end-to-end joint optimization manner such that the privacy lens system 202 and the network 200 are co-optimized to ensure both privacy preservation and accurate depression detection.

In one example, one advantage of co-optimized learning/training is to minimize a combination of losses that guide the system to capture privacy protected images while extracting relevant features.

A total loss function for joint optimization, in one example, is a weighted sum of individual losses from the previous stages as follows:

$${L}_{total} = {λ}_{i} {L}_{i} + {λ}_{e} {L}_{e} + {λ}_{i d} {L}_{i d} + {λ}_{s} {L}_{s} .$$

In this example, λᵢ, λₑ, λ_{id} and λₛ are, respectively, weighting coefficients for identify obfuscation, emotion extraction, depression feature extraction, and final score prediction losses.

In one example, the optimization process is performed using gradient descent in the models, where the parameters of both the learnable lens system 202 and the network 200 are updated iteratively to minimize the total loss. The gradient descent allows important features or relevant features to be identified and preserved by adjusting the learnable parameters of the lens system accordingly. Performing a joint optimization helps ensure that the captured images are unrecognizable in terms of identity but rich in features needed for accurate depression detection.

Embodiments of the invention thus include or relate to a dynamic lens that co-optimizes with a deep network to obscure identity features while preserving depression-relevant data. In contrast to fixed methods, the lens adapts to maintain privacy without sacrificing detection accuracy.

The network (e.g., FFNet) is configured to extract features from privacy-preserving images, ensuring effective depression detection. A staged training method refines the network's focus from identity obfuscation to emotional and depression specific features, enhancing detection accuracy over single-stage methods.

A multimodal transformer effectively merges emotional and depression features, surpassing conventional fusion techniques. An end-to-end joint optimization of the lens system and the deep network ensures a balance between privacy and feature retention.

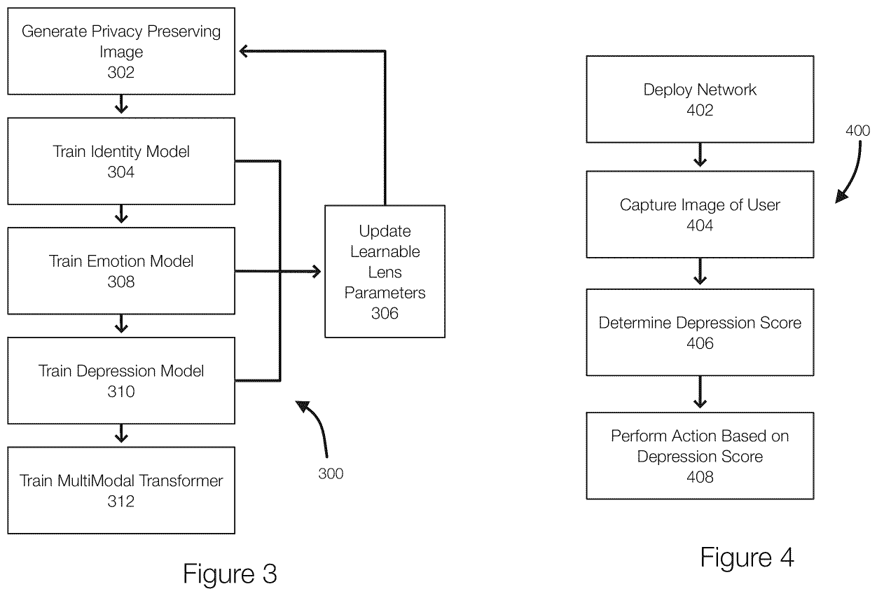

FIG. 3 discloses aspects of a method for detecting a health issue in a user. The method 300 includes generating 302 a privacy preserving image. The privacy preserving image is generated by a learnable privacy lens of a privacy lens system (or privacy camera). For example, the privacy preserving image of a user or of a scene may be blurred in a manner that obscures identity while preserving features or structure related to emotion, depression, or other characteristic or issue.

The privacy preserving image may be provided to an deep network and initially input to train 304 an identity model. In one example, the identity model is trained such that the identity of the user can be determined from the privacy preserving image. The loss or back propagation of the identity model can be used to update 306 the parameters of the learnable lens included in the privacy camera.

The privacy preserving image may then be regenerated based on the updated lens parameters or the privacy preserving image may be simply input without update to train 308 or finetune an emotion model in an emotion stage of the network. The emotion model is configured to extract emotion features and/or identify an emotion expressed in the privacy preserving image. In one example, the emotion model may identify probabilities for each of the features or classes of emotions. Similarly, a loss or back propagation can be used to update 306 the learnable parameters of the privacy camera.

The privacy preserving image may be updated based on the updated parameters or the privacy preserving image may be simply input to the depression stage to train 310 a depression model. The depression model is trained to extract depression features and their probabilities. The loss of the depression model or back propagation may be used to update 306 the learnable parameters of the lens.

The multimodal transformer is then trained 312 using the extracted emotion and depression features to generate a depression score.

Generally, the method 300 is performed to minimize or reduce a total loss. Further, the learnable parameters and models in the stages of the network are optimized in a cooperative manner.

FIG. 4 discloses aspects of monitoring user health. The method 400 may include deploying 402 a trained network. This may include updating privacy cameras with the learned parameters and the like. As a result, the privacy cameras for multiple users can capture 404 privacy preserving images. The privacy preserving images are then input to the network and a depression score is determined 406 for each of the users. Actions are then performed 408 based on the depression score.

If the depression score is above a threshold score, for example, a user may be referred to an appropriate doctor, the user may be encouraged to visit a doctor, take advantage of available amenities, take advantage of resources offered by an entity. The intervention or action performed may depend on the score. Embodiments of the invention allow an entity to take proactive actions to detect the beginning stages of depression and help their users or employees be more productive and more healthy.

Embodiments, such as the examples disclosed herein, may be beneficial in a variety of respects. For example, and as will be apparent from the present disclosure, one or more embodiments may provide one or more advantageous and unexpected effects, in any combination, some examples of which are set forth below. It should be noted that such effects are neither intended, nor should be construed, to limit the scope of the claims in any way. It should further be noted that nothing herein should be construed as constituting an essential or indispensable element of any embodiment. Rather, various aspects of the disclosed embodiments may be combined in a variety of ways so as to define yet further embodiments. For example, any element(s) of any embodiment may be combined with any element(s) of any other embodiment, to define still further embodiments. Such further embodiments are considered as being within the scope of this disclosure. As well, none of the embodiments embraced within the scope of this disclosure should be construed as resolving, or being limited to the resolution of, any particular problem(s). Nor should any such embodiments be construed to implement, or be limited to implementation of, any particular technical effect(s) or solution(s). Finally, it is not required that any embodiment implement any of the advantageous and unexpected effects disclosed herein.

The following is a discussion of aspects of example operating environments for various embodiments. This discussion is not intended to limit the scope of the claims or this disclosure, or the applicability of the embodiments, in any way.

In general, embodiments may be implemented in connection with systems, software, and components, that individually and/or collectively implement, and/or cause the implementation of, health monitoring operations, depression detection operations, co-optimizing operations for learnable lens and a deep network, emotion detection operations, privacy preserving operations, and the like or combinations thereof. More generally, the scope of this disclosure embraces any operating environment in which the disclosed concepts may be useful.

New and/or modified data collected and/or generated in connection with some embodiments, may be stored in a data storage environment that may take the form of a public or private cloud storage environment, an on-premises storage environment, and hybrid storage environments that include public and private elements. Any of these example storage environments, may be partly, or completely, virtualized. The storage environment may comprise, or consist of, a datacenter which is operable to perform operations initiated by one or more clients or other elements of the operating environment.

Example cloud computing environments, which may or may not be public, include storage environments that may provide data protection functionality for one or more clients. Another example of a cloud computing environment is one in which processing, data protection, and other, services may be performed on behalf of one or more clients. More generally however, the scope of this disclosure is not limited to employment of any particular type or implementation of cloud computing environment.

In addition to the cloud environment, the operating environment may also include one or more clients that are capable of collecting, modifying, and creating, data. As such, a particular client may employ, or otherwise be associated with, one or more instances of each of one or more applications that perform such operations with respect to data. Such clients may comprise physical machines, containers, or virtual machines (VMs).

Particularly, devices in the operating environment may take the form of software, physical machines, containers, or VMs, or any combination of these, though no particular device implementation or configuration is required for any embodiment. Similarly, data storage system components such as databases, storage servers, storage volumes (LUNs), storage disks, servers and clients, for example, may likewise take the form of software, physical machines, containers, or virtual machines (VMs), though no particular component implementation is required for any embodiment. Where VMs are employed, a hypervisor or other virtual machine monitor (VMM) may be employed to create and control the VMs. The term VM embraces, but is not limited to, any virtualization, emulation, or other representation, of one or more computing system elements, such as computing system hardware. A VM may be based on one or more computer architectures, and provides the functionality of a physical computer. A VM implementation may comprise, or at least involve the use of, hardware and/or software. An image of a VM may take the form of a .VMX file and one or more .VMDK files (VM hard disks) for example.

As used herein, the terms ‘object’ and ‘data’ are intended to be broad in scope. Example embodiments are applicable to any system capable of storing and handling various types of objects or data, in analog, digital, or other form.

It is noted that any operation(s) of any of the methods disclosed herein, may be performed in response to, as a result of, and/or, based upon, the performance of any preceding operation(s). Correspondingly, performance of one or more operations, for example, may be a predicate or trigger to subsequent performance of one or more additional operations. Thus, for example, the various operations that may make up a method may be linked together or otherwise associated with each other by way of relations such as the examples just noted. Finally, and while it is not required, the individual operations that make up the various example methods disclosed herein are, in some embodiments, performed in the specific sequence recited in those examples. In other embodiments, the individual operations that make up a disclosed method may be performed in a sequence other than the specific sequence recited.

Following are some further example embodiments. These are presented only by way of example and are not intended to limit the scope of this disclosure or the claims in any way.

Embodiment 1. A method comprising: generating a privacy preserving image of a user at a privacy lens system, training an identity model of a deep network based on the privacy preserving image in a training stage and updating learnable parameters of a learnable lens included in the privacy lens system based on a gradient descent of the identity model, training an emotion model of the deep network based on the privacy preserving image in an emotion stage and updating the learnable parameters of the learnable lens based on a gradient descent of the emotion model, training a depression model of the deep network based on the privacy preserving image in an emotion stage and updating the learnable parameters of the learnable lens based on a gradient descent of the depression model, and inputting emotion features extracted from the privacy preserving image by the emotion model and depression features extracted from the privacy preserving image by the depression model to train a multimodal transformer of the deep network to generate a depression score.

Embodiment 2. The method of embodiment 1, wherein the identity model is configured to determine an identity of the user from the privacy preserving image.

Embodiment 3. The method of embodiment 1 and/or 2, wherein the learnable parameters comprise Zernike polynomials, and wherein the learnable parameters are configured to obscure identity while preserving features or structures in the privacy preserving image related to emotion and/or depression.

Embodiment 4. The method of embodiment 1, 2, and/or 3, wherein the privacy preserving image is updated based on the updated learnable parameters determined from the identity model before training the emotion model, wherein the privacy preserving image is updated based on the updated learnable parameters determined from the emotion model before training the depression model.

Embodiment 5. The method of embodiment 1, 2, 3, and/or 4, wherein the emotion model is trained based on a cross-entropy loss, wherein the identity model is trained based at least on a landmark loss.

Embodiment 6. The method of embodiment 1, 2, 3, 4, and/or 5, wherein the depression model is trained using emotion features extracted by the emotion model as priors.

Embodiment 7. The method of embodiment 1, 2, 3, 4, 5, and/or 6, wherein the multimodal model is configured to fuse the emotion features and the depression features.

Embodiment 8. The method of embodiment 1, 2, 3, 4, 5, 6, and/or 7, wherein the multimodal is trained based on a mean squared error loss based on a predicted depression score and an actual depression score.

Embodiment 9. The method of embodiment 1, 2, 3, 4, 5, 6, 7, and/or 8, wherein the learnable parameters and parameters of the identity model, the emotion model, the depression model, and the multimodal transformer are updated iteratively to minimize a total loss, wherein a total loss is

$${L}_{total} = {λ}_{i} {L}_{i} + {λ}_{e} {L}_{e} + {λ}_{i d} {L}_{i d} + {λ}_{s} {L}_{s} ,$$

wherein λᵢ, λₑ, λ_{id} and λₛ comprise, respectively, weighting coefficients for identify obfuscation, emotion extraction, depression feature extraction, and final score prediction losses.

Embodiment 10. A method for monitoring health of a user, the method comprising: capturing a privacy preserving image of the user with a privacy camera that includes a learnable lens, wherein learnable parameters of the learnable lens are configured such that identity features of the user are obscured in the privacy preserving image while emotion features and depression features for detecting depression are preserved in the privacy preserving image, determining an identity of the user in an identity stage of a deep network, extracting the emotion features from the privacy preserving image in an emotion stage of the deep network, extracting the depression features from the privacy preserving image in the depression stage of the deep network, and fusing the emotion features and the depression features to generate a depression score for the user with a multimodal transformer in a multimodal transformer stage of the deep network, and performing an action to benefit the user when the depression score exceeds a threshold depression score.

Embodiment 11. The method of embodiment 10, further comprising: deploying the deep network to a computing system, wherein identity stage includes an identity model configured to determine the identity of the user from the privacy preserving image, the emotion stage includes an emotion model configured to extract the emotion features from the privacy preserving image, the detection stage includes a depression model configured to extract the depression features from the privacy preserving image, and the multimodal transformer stage includes a multimodal transformer configured to fuse the emotion features and the depression features to generate a depression score for the user.

Embodiment 12. A system, comprising hardware and/or software, operable to perform any of the operations, methods, or processes, or any portion of any of these, disclosed herein.

Embodiment 13. A non-transitory storage medium having stored therein instructions that are executable by one or more hardware processors to perform operations comprising the operations of any one or more of embodiments 1-11.

The embodiments disclosed herein may include the use of a special purpose or general-purpose computer including various computer hardware or software modules, as discussed in greater detail below. A computer may include a processor and computer storage media carrying instructions that, when executed by the processor and/or caused to be executed by the processor, perform any one or more of the methods disclosed herein, or any part(s) of any method disclosed.

As indicated above, embodiments within the scope of this disclosure also include computer storage media, which are physical media for carrying or having computer-executable instructions or data structures stored thereon. Such computer storage media may be any available physical media that may be accessed by a general purpose or special purpose computer.

By way of example, and not limitation, such computer storage media may comprise hardware storage such as solid state disk/device (SSD), RAM, ROM, EEPROM, CD-ROM, flash memory, phase-change memory (“PCM”), or other optical disk storage, magnetic disk storage or other magnetic storage devices, or any other hardware storage devices which may be used to store program code in the form of computer-executable instructions or data structures, which may be accessed and executed by a general-purpose or special-purpose computer system to implement the disclosed functionality. Combinations of the above should also be included within the scope of computer storage media. Such media are also examples of non-transitory storage media, and non-transitory storage media also embraces cloud-based storage systems and structures, although the scope of this disclosure is not limited to these examples of non-transitory storage media.

Computer-executable instructions comprise, for example, instructions and data which, when executed, cause a general purpose computer, special purpose computer, or special purpose processing device to perform a certain function or group of functions. As such, some embodiments may be downloadable to one or more systems or devices, for example, from a website, mesh topology, or other source. As well, the scope of this disclosure embraces any hardware system or device that comprises an instance of an application that comprises the disclosed executable instructions.

Although the subject matter has been described in language specific to structural features and/or methodological acts, it is to be understood that the subject matter defined in the appended claims is not necessarily limited to the specific features or acts described above. Rather, the specific features and acts disclosed herein are disclosed as example forms of implementing the claims.

As used herein, the term module, component, client, agent, service, engine, or the like may refer to software objects or routines that execute on the computing system. These may be implemented as objects or processes that execute on the computing system, for example, as separate threads. While the system and methods described herein may be implemented in software, implementations in hardware or a combination of software and hardware are also possible and contemplated. In the present disclosure, a ‘computing entity’ may be any computing system as previously defined herein, or any module or combination of modules running on a computing system.

In at least some instances, a hardware processor is provided that is operable to carry out executable instructions for performing a method or process, such as the methods and processes disclosed herein. The hardware processor may or may not comprise an element of other hardware, such as the computing devices and systems disclosed herein.

In terms of computing environments, embodiments may be performed in client-server environments, whether network or local environments, or in any other suitable environment. Suitable operating environments for at least some embodiments include cloud computing environments where one or more of a client, server, or other machine may reside and operate in a cloud environment.

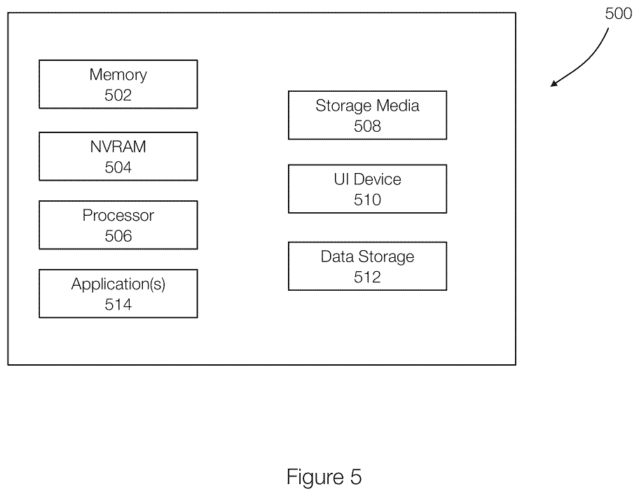

With reference briefly now to FIG. 5, any one or more of the entities disclosed, or implied, by the Figures, and/or elsewhere herein, may take the form of, or include, or be implemented on, or hosted by, a physical computing device, one example of which is denoted at 500. As well, where any of the aforementioned elements comprise or consist of a virtual machine (VM), that VM may constitute a virtualization of any combination of the physical components disclosed in FIG. 5.

In the example of FIG. 5, the physical computing device 500 includes a memory 502 which may include one, some, or all, of random access memory (RAM), non-volatile memory (NVM) 504 such as NVRAM for example, read-only memory (ROM), and persistent memory, one or more hardware processors 506, non-transitory storage media 508, UI device 510, and data storage 512. One or more of the memory components 502 of the physical computing device 500 may take the form of solid state device (SSD) storage. As well, one or more applications 514 may be provided that comprise instructions executable by one or more hardware processors 506 to perform any of the operations, or portions thereof, disclosed herein.

Such executable instructions may take various forms including, for example, instructions executable to perform any method or portion thereof disclosed herein, and/or executable by/at any of a storage site, whether on-premises at an enterprise, or a cloud computing site, client, datacenter, data protection site including a cloud storage site, or backup server, to perform any of the functions disclosed herein. As well, such instructions may be executable to perform any of the other operations and methods, and any portions thereof, disclosed herein. Embodiments of the invention may be implemented in a distributed manner as well.

The described embodiments are to be considered in all respects only as illustrative and not restrictive. All changes which come within the meaning and range of equivalency of the claims are to be embraced within their scope.

What is claimed is:

1. A method comprising:
generating a privacy preserving image of a user at a privacy lens system;
training an identity model of a deep network based on the privacy preserving image in a training stage and updating learnable parameters of a learnable lens included in the privacy lens system based on a gradient descent of the identity model;
training an emotion model of the deep network based on the privacy preserving image in an emotion stage and updating the learnable parameters of the learnable lens based on a gradient descent of the emotion model;
training a depression model of the deep network based on the privacy preserving image in an emotion stage and updating the learnable parameters of the learnable lens based on a gradient descent of the depression model; and
inputting emotion features extracted from the privacy preserving image by the emotion model and depression features extracted from the privacy preserving image by the depression model to train a multimodal transformer of the deep network to generate a depression score.

2. The method of claim 1, wherein the identity model is configured to determine an identity of the user from the privacy preserving image.

3. The method of claim 1, wherein the learnable parameters comprise Zernike polynomials, and wherein the learnable parameters are configured to obscure identity while preserving features or structures in the privacy preserving image related to emotion and/or depression.

4. The method of claim 3, wherein the privacy preserving image is updated based on the updated learnable parameters determined from the identity model before training the emotion model, wherein the privacy preserving image is updated based on the updated learnable parameters determined from the emotion model before training the depression model.

5. The method of claim 1, wherein the emotion model is trained based on a cross-entropy loss, wherein the identity model is trained based at least on a landmark loss.

6. The method of claim 1, wherein the depression model is trained using emotion features extracted by the emotion model as priors.

7. The method of claim 1, wherein the multimodal model is configured to fuse the emotion features and the depression features.

8. The method of claim 7, wherein the multimodal is trained based on a mean squared error loss based on a predicted depression score and an actual depression score.

9. The method of claim 1, wherein the learnable parameters and parameters of the identity model, the emotion model, the depression model, and the multimodal transformer are updated iteratively to minimize a total loss, wherein a total loss is Lₜₒₜₐₗ=λᵢLᵢ+λₑLₑ+λ_{id}L_{id}+λₛLₛ, wherein λᵢ, λₑ, λ_{id} and λₛ comprise, respectively, weighting coefficients for identify obfuscation, emotion extraction, depression feature extraction, and final score prediction losses.

10. A non-transitory storage medium having stored therein instructions that are executable by one or more hardware processors to perform operations comprising:
generating a privacy preserving image of a user at a privacy lens system;
training an identity model of a deep network based on the privacy preserving image in a training stage and updating learnable parameters of a learnable lens included in the privacy lens system based on a gradient descent of the identity model;
training an emotion model of the deep network based on the privacy preserving image in an emotion stage and updating the learnable parameters of the learnable lens based on a gradient descent of the emotion model;
training a depression model of the deep network based on the privacy preserving image in an emotion stage and updating the learnable parameters of the learnable lens based on a gradient descent of the depression model; and
inputting emotion features extracted from the privacy preserving image by the emotion model and depression features extracted from the privacy preserving image by the depression model to train a multimodal transformer of the deep network to generate a depression score.

11. The non-transitory storage medium of claim 10, wherein the identity model is configured to determine an identity of the user from the privacy preserving image.

12. The non-transitory storage medium of claim 10, wherein the learnable parameters comprise Zernike polynomials, and wherein the learnable parameters are configured to obscure identity while preserving features or structures in the privacy preserving image related to emotion and/or depression.

13. The non-transitory storage medium of claim 12, wherein the privacy preserving image is updated based on the updated learnable parameters determined from the identity model before training the emotion model, wherein the privacy preserving image is updated based on the updated learnable parameters determined from the emotion model before training the depression model.

14. The non-transitory storage medium of claim 10, wherein the emotion model is trained based on a cross-entropy loss, wherein the identity model is trained based at least on a landmark loss.

15. The non-transitory storage medium of claim 10, wherein the depression model is trained using emotion features extracted by the emotion model as priors.

16. The non-transitory storage medium of claim 10, wherein the multimodal model is configured to fuse the emotion features and the depression features.

17. The non-transitory storage medium of claim 16, wherein the multimodal is trained based on a mean squared error loss based on a predicted depression score and an actual depression score.

18. The non-transitory storage medium of claim 10, wherein the learnable parameters and parameters of the identity model, the emotion model, the depression model, and the multimodal transformer are updated iteratively to minimize a total loss, wherein a total loss is Lₜₒₜₐₗ=λᵢLᵢ+λₑLₑ+λ_{id}L_{id}+λₛLₛ, wherein λᵢ, λₑ, λ_{id} and λₛ comprise, respectively, weighting coefficients for identify obfuscation, emotion extraction, depression feature extraction, and final score prediction losses.

19. A method for monitoring health of a user, the method comprising:
capturing a privacy preserving image of the user with a privacy camera that includes a learnable lens, wherein learnable parameters of the learnable lens are configured such that identity features of the user are obscured in the privacy preserving image while emotion features and depression features for detecting depression are preserved in the privacy preserving image;
determining an identity of the user in an identity stage of a deep network;
extracting the emotion features from the privacy preserving image in an emotion stage of the deep network;
extracting the depression features from the privacy preserving image in the depression stage of the deep network; and
fusing the emotion features and the depression features to generate a depression score for the user with a multimodal transformer in a multimodal transformer stage of the deep network; and
performing an action to benefit the user when the depression score exceeds a threshold depression score.

20. The method of claim 1, further comprising:
deploying the deep network to a computing system, wherein identity stage includes an identity model configured to determine the identity of the user from the privacy preserving image, the emotion stage includes an emotion model configured to extract the emotion features from the privacy preserving image, the detection stage includes a depression model configured to extract the depression features from the privacy preserving image, and the multimodal transformer stage includes a multimodal transformer configured to fuse the emotion features and the depression features to generate a depression score for the user.
